(19) United States
(12) Patent Application Publication
(10) Pub. No.: US 2026/0174904 A1
(43) Pub. Date: Jun. 25, 2026

(54) BOB1-seRNA AND APPLICATION THEREOF

(71) Applicant: Tongji Hospital affiliated to Tongji Medical College of Huazhong University of Science & Technology, Wuhan (CN)

(72) Inventors: Bo Yu, Wuhan (CN); Peng Chen, Wuhan (CN); Hong Wang, Wuhan (CN); Jiqi James Wang, Wuhan (CN); Yujing Su, Wuhan (CN)

(21) Appl. No.: 19/192,361

(22) Filed: Apr. 29, 2025

(30) Foreign Application Priority Data
Dec. 24, 2024 (CN) 202411911556.6

(51) Int. Cl. A61K 48/00; C07K 14/47; C12N 15/86

(52) U.S. Cl. CPC A61K 48/0058; C07K 14/47; C12N 15/86; C12N 2750/14143

(57) ABSTRACT

A B-cell specific octamer-binding protein 1 super-enhancer ribonucleic acid (BOB1-seRNA) and an application thereof are provided. The nucleotide sequence of the BOB1-seRNA is shown in SEQ ID NO: 1. Through experiments, it further confirms that the BOB1-seRNA is downregulated in patients with coronary heart disease and can be used in preparation of a product for diagnosing or predicting the coronary heart disease. The use of this molecular marker can be used for early diagnosis of the coronary heart disease, which is rapid and effective. It is not only of great significance for early treatment of the coronary heart disease and saving medical costs, but also provides therapeutic targets and important basis for clinical applications such as gene therapy and drug therapy.

### CROSS-REFERENCE TO RELATED APPLICATION

This application claims priority to Chinese Patent Application No. 202411911556.6, filed Dec. 24, 2024, which is herein incorporated by reference in its entirety.

### TECHNICAL FIELD

The disclosure relates to the technical field of biomedicines, and more particularly to a B-cell specific octamer-binding protein 1 super-enhancer ribonucleic acid (BOB1-seRNA) and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25017THXT-USP1-US-2025-0014-SL.xml. The XML file is 2,452 bytes; is created on Apr. 24, 2025; and is being submitted electronically via patent center.

### BACKGROUND

Atherosclerosis is a chronic inflammatory disease caused by abnormal deposition of lipids in the arterial wall and is a main cause of cardiovascular and cerebrovascular diseases and their associated mortality. Studies have shown that lipid-regulating therapies through statins and cholesterol absorption inhibitors can significantly reduce risks of the atherosclerosis and related diseases, but residual risks of the cardiovascular diseases still remain. Relevant studies have shown that BOB1 protein plays a key role in reverse cholesterol transport and participates in the development of atherosclerosis-related diseases.

In addition, novel RNA-targeted lipid-lowering drugs have shown remarkable efficacy in regulating lipid homeostasis, but their epigenetic mechanisms underlying cholesterol homeostasis maintenance remain unclear.

### SUMMARY

To solve problems existing in the related art, a purpose of the disclosure is to provide a BOB1-seRNA and an application thereof.

To achieve the above purpose, the disclosure provides the following technical solutions.

In a first aspect, the disclosure provides a BOB1-seRNA, and the nucleotide sequence of the BOB1-seRNA is shown in SEQ ID NO: 1.

In a second aspect, the disclosure provides an application of the BOB1-seRNA in preparation of a drug for preventing, alleviating, or treating atherosclerosis.

In a third aspect, the disclosure provides a drug for preventing, alleviating, or treating atherosclerosis, and an active ingredient of the drug includes a substance that promotes an expression of a BOB1-seRNA.

In a fourth aspect, the disclosure provides an application of the BOB1-seRNA in preparation of a product for predicting a risk of coronary heart disease.

Based on the above technical solutions, the disclosure may achieve the following technical effects.

Through transcriptomic sequencing and corresponding bioinformatics analysis methods for patients with the coronary heart disease, the disclosure innovatively provides a non-coding BOB1-seRNA, which is significantly negatively correlated with the risk of the coronary artery disease in the patients with the coronary heart disease and dilated cardiomyopathy. Extensive animal experiments confirm that upregulation of the BOB1-seRNA can effectively reduce blood lipid levels and decrease a number and area of aortic plaques, thereby exerting an anti-atherosclerotic effect. To achieve therapeutic goals, the disclosure applies human BOB1-seRNA to animal experiments, and results show that a synthetic compound of the BOB1-seRNA can significantly improve lipid imbalances and atherosclerotic plaque formation induced by a Western diet, ultimately providing a basis for the development of clinical coronary heart disease treatment drugs and risk prediction products.

### BRIEF DESCRIPTION OF DRAWINGS

In order to more clearly illustrate technical solutions in embodiments of the disclosure or in the related art, attached drawings necessary for the embodiments are briefly introduced below. It is apparent that the attached drawings described below are merely some embodiments of the disclosure. For those skilled in the art, other attached drawings can be obtained based on these attached drawings without making any inventive effort.

FIG. 1 illustrates a schematic diagram of results of in vitro lipid-lowering effects of a BOB1-seRNA. Specifically, in FIG. 1, A illustrates that overexpression of the BOB1-seRNA can significantly upregulate expression of BOB1; B illustrates that the overexpression of the BOB1-seRNA can significantly reduce intracellular lipid accumulation; and C illustrates that the overexpression of the BOB1-seRNA can significantly enhance capacity for reverse cholesterol transport in cells.

FIG. 2 illustrates a schematic diagram of results of lipid-lowering effects and improvement of hepatic steatosis of the BOB1-seRNA in C57BL/C mice. Specifically, in FIG. 2, A illustrates effects of overexpressing the BOB1-seRNA via a recombinant adeno-associated virus 8 (AAV8) vector on plasma lipid levels; and B illustrates effects of overexpressing the BOB1-seRNA via the recombinant AAV8 vector on hepatic lipid deposition.

FIG. 3 illustrates a schematic diagram of results of improvement of atherosclerosis in apolipoprotein E knockout (APOE−/−) mice by the BOB1-seRNA. Specifically, in FIG. 3, A illustrates effects of overexpressing the BOB1-seRNA via the recombinant AAV8 vector on plasma levels of inflammatory factors (interleukin-1 beta abbreviated as IL-1β, interleukin-6 abbreviated as IL-6, monocyte chemoattractant protein-1 abbreviated as MCP-1, and tumor necrosis factor-alpha abbreviated as TNF-α); B illustrates effects of overexpressing the BOB1-seRNA via the recombinant AAV8 vector on an area of aortic atherosclerosis; and C illustrates effects of overexpressing the BOB1-seRNA via the recombinant AAV8 vector on a size of an aortic valvular atherosclerotic plaque.

FIG. 4 illustrates a relationship between an expression level of the BOB1-seRNA and a risk of coronary artery disease in a population of patients with the coronary heart disease. Specifically, in FIG. 4, A illustrates that patients with a higher expression level of the BOB1-seRNA have a lower Gensini score, indicating a lower risk of the coronary artery disease; and B illustrates that the patients with the higher expression level of the BOB1-seRNA have a lower synergy between percutaneous coronary intervention with taxus and cardiac surgery (SYNTAX) score, also indicating the lower risk of the coronary artery disease.

### DETAILED DESCRIPTION OF EMBODIMENTS

The disclosure will now be described in detail with respect to various illustrative embodiments. This detailed description is not to be taken as a limitation of the disclosure, but rather as a more detailed description of certain aspects, features, and embodiments of the disclosure.

It should be understood that terms used in the disclosure are only for describing specific embodiments and are not intended to limit the disclosure. In addition, for the numerical range in the disclosure, it should be understood that each intermediate value between the upper and lower limits of the range is also specifically disclosed. Any intermediate value within any stated value or range, as well as any smaller range between any other stated value or intermediate value within the range, are also included in the disclosure. These smaller upper and lower limits can be independently included or excluded within the range.

Unless otherwise indicated, all technical and scientific terms used herein have the same meaning as commonly understood by those skilled in the art to which the disclosure belongs. Although only specific methods and materials are described in the disclosure, any methods and materials similar or equivalent to those described herein may also be used in the practice or testing of the disclosure. All publications mentioned herein are incorporated by reference to disclose and describe the methods and/or materials in connection with the publications. In the event of a conflict between any incorporated publications and the content of this specification, the content of this specification shall prevail.

It is apparent to those skilled in the art that various improvements and modifications can be made to the specific embodiments described in the specification of the disclosure without departing from the scope or spirit of the disclosure. Other embodiments that can be derived from the specification of the disclosure would also be apparent to those skilled in the art. The specification and embodiments of this application are merely illustrative.

The terms “comprising”, “including”, “having”, “containing” and the like, as used in this text, are open-ended terms, meaning they include but are not limited to.

The technical solutions described in the disclosure, unless specifically stated otherwise, are conventional solutions in the field. The reagents or materials used, unless specifically stated otherwise, are commercially available or publicly disclosed.

An embodiment of the disclosure provides a BOB1-seRNA. The nucleotide sequence of the BOB1-seRNA is shown in SEQ ID NO: 1.

Another embodiment of the disclosure provides an application of the BOB1-seRNA in preparation of a drug for preventing, alleviating, or treating atherosclerosis.

Still another embodiment of the disclosure provides a drug for preventing, alleviating, or treating atherosclerosis. An active ingredient of the drug includes a substance that promotes an expression of a BOB1-seRNA.

In some specific embodiments, the substance that promotes the expression of the BOB1-seRNA includes a recombinant expression vector for overexpressing the BOB1-seRNA, a super-expression vector, an interference vector, a recombinant virus, or a recombinant gene expression kit.

In some specific embodiments, the drug further includes a pharmaceutically acceptable excipient.

In some specific embodiments, a dosage form of the drug includes an oral preparation, a topical preparation, an injectable preparation, or a molecular reagent kit.

Even still another embodiment of the disclosure provides an application of the BOB1-seRNA in preparation of a product for predicting a risk of coronary heart disease.

The disclosure further confirms through experiments that the expression of the BOB1-seRNA is downregulated in patients with the coronary heart disease, and it can be used in the preparation of the product for diagnosing or predicting the coronary heart disease. This molecular marker (i.e., the BOB1-seRNA) can be utilized for early diagnosis of the coronary heart disease, which is rapid and effective. It is not only of great significance for early treatment of the coronary heart disease and saving of medical costs, but also provides a therapeutic target and an important basis for clinical applications such as gene therapy and drug therapy.

### Embodiment 1

A study targets 380 patients with coronary heart disease who visits a tertiary hospital in Hubei Province during the period from 2020 to 2022, including 211 males and 169 females with ages ranging from 18 years to 70 years. Peripheral blood samples are collected from these patients for transcriptomic sequencing, and results are subsequently analyzed using bioinformatics websites (National Center for Biotechnology Information and EMBL's European Bioinformatics Institute).

A full-length sequence of the BOB1-seRNA is amplified using a rapid amplification of complementary deoxyribonucleic acid 5′end (5′RACE) method and a rapid amplification of complementary deoxyribonucleic acid 3′end (3′RACE) method, as shown below:

SEQ ID NO: 1:
AGCTCAGGGCCCTCCGAACAAAGCAGCGCTCGGTGCCGGACCTGCAGCTC
TCCCCACCCGGCAGCAGCGCCCTCAGCACGCCCGGGAACCCCCGACCCCT
CTGAGCCCGGGGTACTGCGCCCGGGTCTCCACGCCCAGAGATGCTCCCCG
GTCTCCACCGTCGGGCAAGCCCCAAGCGCAGCAGCGCAGAGTCCTGGGGT
CACCAGAGCTCGTACTAGGACATCGTCTCCCCATTTAACACCGCCTCCGG
TCCCATCTGAGTTGCAAGTGGTGGGGATGTGGGGCTCCGGATCAAAGTCC
CCGAAACCGAGCACTTCCCGAAGCCTCCTTGGCCTCGAAACAAAACAATA
ACGCCCAAGCGACCTACTGCCTTGGAGATTCCCAGCCTGAGCTCCCCAGG
TCCCTCAGAGGACCCGAGAGGAACATCTTACTGCTTCTCTTCATAAATTG
AGAGGAAGGAGGCCACAAA.

Specific sequence details of the BOB1-seRNA can be found via NONCODE TRANSCRIPT ID: NONHSAT133803.2.

### 1. Cell Culture

Tohoku hospital pediatrics-1 (THP-1) cell line is a type of human monocytic leukemia cell that grows in suspension. THP-1 cells from the THP-1 cell line are cultured in a culture flask containing sterile Roswell Park Memorial Institute 1640 (RPMI 1640) complete medium supplemented with 10% fetal bovine serum and 1% penicillin-streptomycin to prevent contamination. The culture flask is incubated in a humidified incubator at 37° C. with a carbon dioxide (CO₂) concentration of 5%. When the THP-1 cells reach a logarithmic growth phase, the THP-1 cells can be passaged for subsequent experimental use.

### 2. Plasmid Construction

Using human complementary deoxyribonucleic acid (cDNA) as a template, the full-length sequence of the BOB1-seRNA is amplified by a polymerase chain reaction (PCR) to obtain a PCR product. To clone the PCR product into a pcDNA3.1 vector to obtain a recombinant plasmid, Kpn I and Mlu I restriction enzyme sites are incorporated into forward and reverse primers of the PCR product, respectively. For identification whether the recombinant plasmid is constructed correctly, the recombinant plasmid solution and a positive clone bacterial solution are sent to Wuhan Aoke Company for Sanger sequencing, confirming that the recombinant plasmid is constructed correctly.

### 3. Cell Transfection

\textcircled{1}. Passage: after digesting the THP-1 cells, the THP-1 cells are inoculated into a 24-well cell culture plate. When the THP-1 cells reach 50%-70% confluence, they are ready for transfection.

\textcircled{2}. A mixture of 9 microliters (μL) of transfection reagent from Thermo Fisher Scientific, 200 μL of serum-free culture medium, and 10 μL of plasmid vector to be transfected is prepared. The mixture is homogenized and left at room temperature for later use.

\textcircled{3}. Old culture medium of THP-1 cells to be transfected is removed and replaced with new culture medium.

\textcircled{4}. The mixture prepared in the step \textcircled{2} is added to the THP-1 cells to be transfected.

\textcircled{5}. The new culture medium is changed three days later. At this point, the THP-1 cells are successful transfected, thereby obtaining transfected cells. Subsequent experiments can be conducted.

4. Quantitative PCR (qPCR) Experiment
(1) RNA Extraction from Cells

\textcircled{1}. Approximately 100 μL of the transfected cells are resuspended in 1 milliliter (mL) of Trizol reagent, vortexed for 10 seconds, and then incubated at room temperature for 5 minutes. Subsequently, 200 μL (i.e., ⅕ of the volume of the Trizol reagent) of chloroform (i.e., trichloromethane) is added and mixed thoroughly to obtain a mixture. The mixture is allowed to stand for 15 minutes and then centrifuged at 4° C. for 10 minutes at 12,000 revolutions per minute (rpm). An aqueous phase of the mixture is carefully transferred to a new Eppendorf (EP) tube to obtain a mixture without the aqueous phase. Anhydrous ethanol with a volume 2.5 times that of the aqueous phase or isopropanol with an equal volume of the aqueous phase (RNA is insoluble in the anhydrous ethanol or isopropanol, thus precipitating RNA) is added to the mixture without the anhydrous phase, followed by mixing to obtain a sample. The sample is placed at −20° C./−80° C. for precipitation for over 1 hour to obtain a precipitated sample.

The precipitated sample is centrifuged at 4° C. and 12000 rpm for 15 minutes. The supernatant of the precipitated is discarded, leaving a pallet. The pallet is washed once with pre-chilled ethanol with a volume concentration of 75%, and the supernatant is removed. The last drop of the supernatant is carefully removed using a 10 μL pipette tip, and the pallet is air-dried at room temperature for about 5 minutes.

### (2) Reverse Transcription: CDNA Synthesis

\textcircled{1}. A reverse transcription reagent from Vazyme Biotech Limited Company is added to 10 μL of RNA extracted from the step (1) to obtain a mixture. The mixture is subjected to a reverse transcription program on a PCR instrument to synthesize cDNA.

(3) qPCR

\textcircled{1}. The cDNA obtained in the step (2) is mixed with a qPCR reagent and corresponding qPCR primers from the Vazyme Biotech Limited Company, followed by performing the qPCR experiment using the qPCR instrument, thereby obtaining a qPCR result.

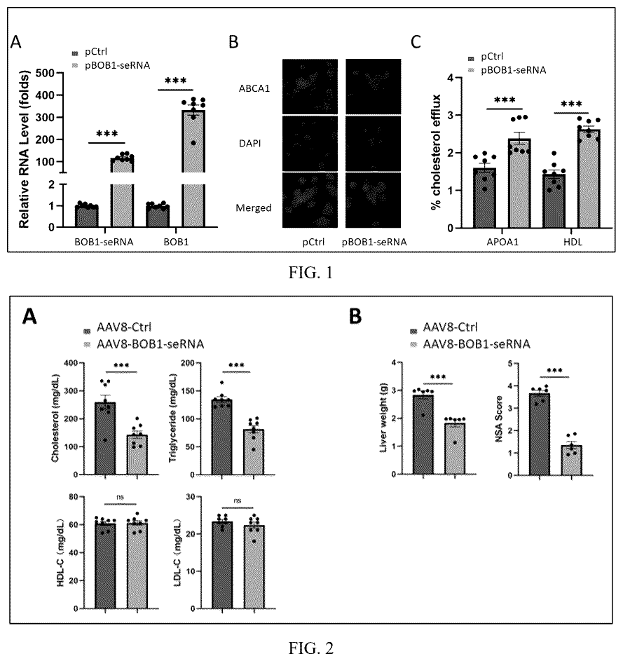

The qPCR result indicates that overexpression of the BOB1-seRNA significantly upregulates an expression of BOB1 (as shown in A in FIG. 1).

### 5. Immunofluorescence Staining

\textcircled{1}. THP-1 cells overexpressing the BOB1-seRNA as an experimental group and unmanipulated THP-1 cells as a control group are inoculated into 24-well plates containing sterile cell-attached slides, respectively. The above two types of THP-1 cells are then respectively induced to differentiate into adherent cells with 100 nanomoles per liter (nM) phorbol 12-myristate 13-acetate (PMA).

\textcircled{2}. Oxidized low-density lipoprotein (Ox-LDL) is added to culture medium of the experimental group to a final concentration of 50 nanograms per milliliter (ng/ml) for 24 hours to induce cell foaming. The control group is not treated with ox-LDL.

\textcircled{3}. After ox-LDL incubation, cells in each group are washed three times with 1× phosphate buffered saline (1×PBS). Then, 500 μL of 10% neutral buffered formalin (NBF) is added to each well for fixation for 30 minutes.

\textcircled{4}. Fixative (i.e., the NBF) of each group is discarded, and an apolipoprotein A-1 (ApoA-1) staining solution is added for staining in the dark for 30 minutes to obtain stained cells.

\textcircled{5}. The stained cells are washed three times with isopropanol with a volume concentration of 60% for decolorization.

\textcircled{6}. Nuclei of each group are stained with fluorescent dye for 30 seconds, followed by three washes with 1×PBS (optional).

\textcircled{7}. Cell-attached slides of each group are placed on glass slides and sealed with glycerol gelatin. The cells are then observed and photographed under a fluorescence microscope.

A result of the immunofluorescence staining experiment indicates that the overexpression of the BOB1-seRNA significantly reduces intracellular lipid accumulation (as shown in B in FIG. 1).

### 6. Cholesterol Efflux Assay

Cholesterol efflux assay, a most common method for detecting a cholesterol efflux rate, is performed as follows.

\textcircled{1}. Unmanipulated THP-1 cells, used as a control group, are inoculated into a 24-well cell culture plate at a density of 2.5×10⁵ cells per well, and cultured in a cell culture incubator at 37° C., 5% CO₂, and appropriate humidity.

\textcircled{2}. After 24 hours, the unmanipulated THP-1 cells are treated with 100 nM PMA for 48 hours to induce differentiation into adherent cells.

\textcircled{3}. Waste liquid is discarded, the adherent cells are washed twice with 1×PBS, and then 500 μL of 5 micromoles per liter (μM) naphtalimide (NBD) cholesterol medium without phenol red is added to each well for overnight incubation in the cell culture incubator.

\textcircled{4}. The next day, waste liquid is removed, the adherent cells after incubation are washed twice with 1×PBS, and then 500 μL of mixed solution containing high-density lipoprotein (HDL), ApoA-1, a standard serum efflux-inducing solution, and a synchronization solution are added to each well for further incubation for 4 hours in the cell culture incubator.

\textcircled{5}. After incubation, cell supernatant is collected into a new brown light-protected EP tube with a volume of 1.5 mL. Subsequently, 100 μL of the cell supernatant is added to each well of a black 96-well plate, and fluorescence intensity is measured using a fluorescence microplate reader with an excitation wavelength of 469 nanometers (nm) and an emission wavelength of 537 nm.

\textcircled{6}. The adherent cells are washed twice with 1×PBS, then lysed with 500 μL of 0.1% Triton X-100 and shaken for 10 minutes on a horizontal shaker to obtain cell lysate. The cell lysate is homogenized with a pipette, transferred to a new brown light-protected EP tube with a volume of 1.5 mL, and centrifuged at 12,000 times acceleration of gravity (12,000 g) for 10 minutes. The cell lysate is then added to ae black 96-well plate at 100 μL per well, and fluorescence intensity of the cell lysate is measured.

\textcircled{7}. The cholesterol efflux rate is calculated using the following formula: the cholesterol efflux rate is calculated as a fluorescence value of the cell supernatant divided by a sum of the fluorescence value of the cell supernatant and a fluorescence value of the cell lysate, multiplied by 100%.

The BOB1-seRNA is constructed into the pcDNA3.1 plasmid vector, and the above steps are repeated. Specific steps are as follows.

\textcircled{1}. THP-1 cells transfected with the pcDNA3.1 plasmid vector containing the BOB1-seRNA, used as an experimental group, are inoculated into a 24-well cell culture plate at a density of 2.5×10⁵ cells per well, and cultured in a cell culture incubator at 37° C., 5% CO₂, and appropriate humidity.

\textcircled{2}. After 24 hours, the THP-1 cells are treated with 100 nM PMA for 48 hours to induce differentiation into adherent cells.

\textcircled{3}. Waste liquid is discarded, the adherent cells are washed twice with 1×PBS, and then 500 μL of 5 μM NBD cholesterol medium without phenol red is added to each well for overnight incubation in the cell culture incubator.

\textcircled{4}. The next day, waste liquid is removed, the adherent cells after incubation are washed twice with 1×PBS, and then 500 μL of mixed solution containing HDL, ApoA-1, a standard serum efflux-inducing solution, and a synchronization solution are added to each well for further incubation for 4 hours in the cell culture incubator.

\textcircled{5}. After incubation, cell supernatant is collected into a new brown light-protected EP tube with a volume of 1.5 mL. Subsequently, 100 μL of the cell supernatant is added to each well of a black 96-well plate, and fluorescence intensity is measured using the fluorescence microplate reader with the excitation wavelength of 469 nm and the emission wavelength of 537 nm.

\textcircled{6}. The adherent cells are washed twice with 1×PBS, then lysed with 500 μL of 0.1% Triton X-100 and shaken for 10 minutes on a horizontal shaker to obtain cell lysate. The cell lysate is homogenized with a pipette, transferred to a new brown light-protected EP tube with a volume of 1.5 mL, and centrifuged at 12,000 g for 10 minutes. The cell lysate is then added to a black 96-well plate at 100 μL per well, and fluorescence intensity of the cell lysate is measured.

\textcircled{7}. The cholesterol efflux rate is calculated using the following formula: the cholesterol efflux rate is calculated as a fluorescence value of the cell supernatant divided by a sum of the fluorescence value of the cell supernatant and a fluorescence value of the cell lysate, multiplied by 100%.

The cholesterol efflux assay indicates that the overexpression of the BOB1-seRNA significantly enhances capacity of cellular reverse cholesterol transport (as shown in C in FIG. 1).

### 7. Enzyme-Linked Immunosorbent Assay (ELISA)

ELISA is performed using a test kit provided by Xinbosheng Company to detect inflammatory markers (IL-1B, IL-6, MCP-1, and TNF-α) in THP-1 cells overexpressing BOB1-seRNA and in unmanipulated THP-1 cells. Reagents mentioned in this experiment (i.e., the ELISA) all come from the reagent kit. Specific steps are as follows.

\textcircled{1}. Standards are diluted into 8 recommended concentration gradients using the standard/sample diluent according to test kit instructions, and a blank well is set. 100 μL of test samples and diluted standards are added to each well except the blank well, while the blank well is only added with the diluent. The ELISA plate is sealed with a plate sealer and incubated at 37° C. in the dark for 90 minutes.

\textcircled{2}. After incubation, the waste liquid in the ELISA plate is removed by flicking and drying. 350 μL of 1× wash solution is added to each well, then flicked and dried after a 30-second stand.

\textcircled{3}. The step (2) is repeated for a total of 5 washes.

\textcircled{4}. 100 μL of diluent (i.e., PBS) without biotinylated antibody is added to the control well, and 100 μL of working solution containing 1× biotinylated antibody is added to remaining wells. The ELISA plate is sealed and incubated at 37° C. in the dark for 60 minutes.

\textcircled{5}. After incubation, the waste liquid is removed by flicking and drying. 350 μL of 1× wash solution is added to each well, then flicked and dried after a 30-second stand.

\textcircled{6}. The step 5 is repeated for a total of 5 washes.

\textcircled{7}. 100 μL of enzyme conjugate diluent is added to the control well, and 100 μL of 1× enzyme conjugate working solution is added to the remaining wells. The ELISA plate is sealed and incubated at 37° C. in the dark for 30 minutes.

\textcircled{8}. After incubation, the waste liquid is removed by flicking and drying. 350 μL of 1× wash solution is added to each well, then flicked and dried after a 30-second stand.

\textcircled{9}. The step \textcircled{8} is repeated for a total of 5 washes.

\textcircled{10}. 100 μL of chromogen substrate (tetramethylbenzidine abbreviated as TMB) is added to each well and incubated at 37° C. in the dark for 15 minutes.

\textcircled{11}. After incubation, the waste liquid was removed by flicking and drying. 350 μL of 1× wash solution is added to each well, then flicked and dried after a 30-second stand.

\textcircled{12}. The step \textcircled{11} is repeated for a total of 5 washes.

\textcircled{13}. 100 μL of stop solution (sulfuric acid with a concentration of 2 moles per liter (M)) is added to each well and mixed. The absorbance (OD value) is measured using a microplate reader at 450 nm within 3 minutes after adding a stop solution to ensure accuracy.

\textcircled{14}. A standard curve is plotted using the standards of different concentrations to calculate exact concentrations of the test samples.

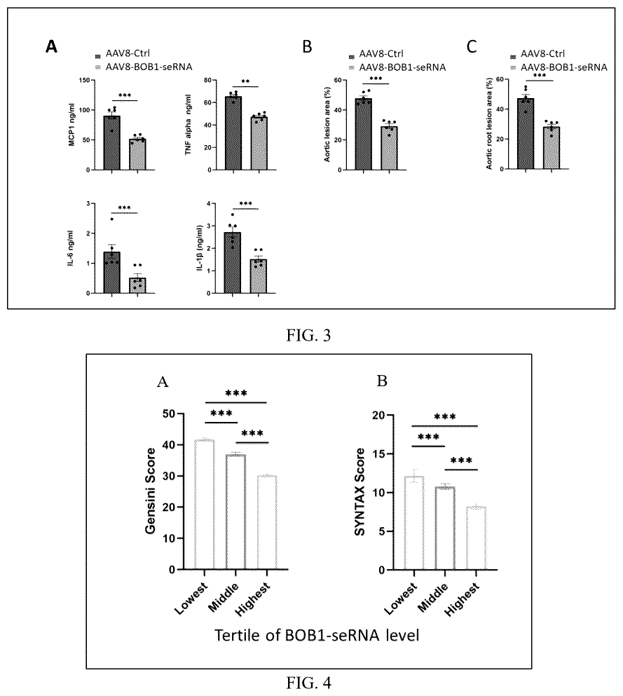

As shown in A in FIG. 3, the levels of inflammatory markers (IL-1B, IL-6, MCP-1, and TNF-α) in the THP-1 cells overexpressing BOB1-seRNA are significantly lower than those in the control group (i.e., the unmanipulated THP-1 cells).

### 8. Paraffin Embedding and Sectioning of Liver Tissue

\textcircled{1} After the model is established, mice are anesthetized via intraperitoneal injection. Thoracic and abdominal aorta tissues are then excised and placed in formaldehyde fixative solution for fixation at room temperature for 48 hours to obtain fixed aorta tissues.

\textcircled{2} Dehydration is carried out on the fixed aorta tissues in the following sequence: ethanol solution with a volume concentration of 50% for 1 hour→ethanol solution with a volume concentration of 70% for 1 hour→ethanol solution with a volume concentration of 80% for 1 hour→ethanol solution with a volume concentration of 90% for 0.5 hours→ethanol solution with a volume concentration of 95% for 0.5 hours→anhydrous ethanol for 0.5 hours→anhydrous ethanol for 0.5 hours.

\textcircled{3} Clarification is performed using xylene solution for 0.5 hours→xylene solution for 0.5 hours.

\textcircled{4} Infiltration is performed with 100% paraffin for 0.5 hours→100% paraffin for 0.5 hours in an oven at 60° C.

\textcircled{5} Embedding is conducted by placing the processed tissue into a mold filled with paraffin, securing the position of the aorta, and cooling it on the cold plate of an embedding machine.

Sectioning is performed by placing the embedded tissue block into the slot of a microtome, cutting it into slices of 5 micrometers (μm) thickness, sealing the slices onto glass slides, and drying them in a 60° C. oven for 0.5 hour.

### 9. Hematoxylin and Eosin (HE) Staining of Slices

\textcircled{1}. Dewaxing: the slices are placed in an oven at 75° C. for 2 hours. The dewaxing procedure is then carried out in the following order: xylene solution for 15 minutes→xylene solution for 15 minutes→xylene solution for 15 minutes→anhydrous ethanol for 5 minutes→anhydrous ethanol for 5 minutes→ethanol solution with a volume concentration of 95% for 120 seconds→ethanol solution with a volume concentration of 85% for 120 seconds→ethanol solution with a volume concentration of 70% for 120 seconds.

\textcircled{2}. Staining: The staining process is carried out as follows: tap water rinse for 1 minute→hematoxylin for 5 minutes→tap water rinse for 5 minutes→differentiation with hydrochloric acid ethanol solution for 3 seconds→tap water rinse for 15 minutes to restore blue color→eosin for 30 seconds.

\textcircled{3}. Dehydration and sealing: The dehydration process is carried out as follows: ethanol solution with a volume concentration of 80% for 120 seconds→ethanol solution with a volume concentration of 90% for 120 seconds→ethanol solution with a volume concentration of 95% for 120 seconds→ethanol solution with a volume concentration of 95% for 120 seconds→anhydrous ethanol for 120 seconds→anhydrous ethanol for 120 seconds→xylene solution for 10 minutes→xylene solution for 5 minutes→sealing with glycerol resin.

\textcircled{4}. Subsequently, an optical microscopy and ImageJ software will be used for further analysis. An HE staining result indicates that the overexpression of the BOB1-seRNA improves hepatic lipid deposition in C57BL/6 mice (as shown in B in FIG. 2)

### 10. Oil Red O Staining

\textcircled{1}. Fixation of slices: frozen slices are placed in a fixative solution containing 10% formaldehyde and fixed at room temperature for 20-30 minutes. A fixation time can be adjusted based on a sample type and experimental requirements.

\textcircled{2}. Washing: The slices are washed with a washing solution (such as an ethanol solution with a volume concentration of 70%) to remove residual fixative. A washing time should be moderate to avoid damaging the slices.

\textcircled{3}. Oil red O staining: The slices are placed in an oil red O staining solution and stained for 5-15 minutes. The slices should be gently agitated during staining to ensure uniform distribution of the dye.

\textcircled{4}. After staining, the slices are washed with a washing solution (such as an ethanol solution with a volume concentration of 70%) to remove excess dye, followed by a 30-second rinse with running water.

\textcircled{5}. Hematoxylin counterstaining: the slices are placed in a hematoxylin solution for counterstaining for 2 minutes. A counterstaining time should not be too long to prevent excessive nuclear staining.

\textcircled{6}. After counterstaining, the slices are rinsed with running water to remove excess dye.

\textcircled{7}. Sealing: The slices are sealed with a sealing agent (such as glycerol gelatin) to prevent fading due to air exposure. The sealing process should ensure that the slices are flat and free of bubbles.

\textcircled{8}. Observation and analysis: the slices are observed under a microscope, and staining results are recorded. Lipids typically appear as deep orange or bright red, and nuclei appear blue-violet. Data analysis is performed based on experimental results to assess lipid content and distribution.

### 11. Packaging and Gene Delivery of AAV8 Virus

The full-length sequence of the BOB1-seRNA is cloned into a pAV-FH vector and sequenced to ensure accuracy. The AAV8 virus is packaged, produced, purified, and titered by Shandong Weizhen Biotechnology. The AAV8 virus carrying BOB1-seRNA gene (1×10¹¹ vector copies) is administered via a single intravenous injection into tail vein. Eight-week-old male C57BL/6 mice are purchased from Beijing Huafukang Biotechnology and acclimated for one week before the experiment. ApoE−/− mice are fed a Western diet for eight weeks before AAV8 injection and then subjected to further experiments. The study is approved by the Institutional Animal Research Committee of Tongji Medical College. Experimental animals are housed in the animal care facilities of Tongji Medical College at 25° C. with a 12/12-hour light/dark cycle, and mice are allowed free access to food and water throughout the study.

C57BL/6 mice (n=12 per group) or ApoE−/− mice (n=10 per group) are randomly divided into rAAV8-Ctrl and rAAV8-BOB1-seRNA groups and fed a Western diet (20 kcal % protein, 20 kcal % carbohydrate, 60 kcal % fat) for 16 weeks. No animals are excluded during the experiment.

After treatment, the mice are subjected to the relevant experiments mentioned above.

Specific experiment and experimental results are as follows. 1, ELISA: in the C57BL/6 mice, the overexpression of the BOB1-seRNA via a recombinant viral vector is shown to reduce plasma triglyceride and cholesterol levels (as shown in A in FIG. 2). 2, liver weight measurement and HE staining: in the C57BL/6 mice, the overexpression of the BOB1-seRNA improves hepatic lipid deposition (as shown in B in FIG. 2). 3, The overexpression of the BOB1-seRNA via the recombinant viral vector and ELISA indicate that in the APOE−/− mice, the overexpression of the BOB1-seRNA via the recombinant viral vector can reduce the release of inflammatory factors (IL-1B, IL-6, MCP-1, TNF-α) in plasma (as shown in A in FIG. 3). 4, Aortic vascular oil red O staining indicates that the overexpression of the BOB1 seRNA in the APOE−/− mice can improve atherosclerosis (as shown in B in FIG. 3). 5. Oil red O staining of aortic valve indicates that the overexpression of the BOB1-seRNA can reduce an area of an atherosclerotic plaque (as shown in C in FIG. 3).

### 12. Isolation of Human Peripheral Blood Mononuclear Cells (PBMCs)

PBMCs from a patient diagnosed with coronary heart disease at a tertiary hospital in Wuhan, Hubei Province, are isolated using a Ficoll separation solution by a density gradient centrifugation method. Specific steps are as follows.

\textcircled{1}. Whole blood is diluted with an equal volume of 1×PBS and gently mixed using a Pasteur pipette to obtain diluted whole blood.

\textcircled{2}. 15 mL of Ficoll separation solution is added to a 50 mL centrifuge tube.

\textcircled{3}. The diluted whole blood is slowly added into the 50 mL centrifuge tube along a tube wall, allowing blood cell suspension to layer on top of the Ficoll separation solution.

\textcircled{4}. The 50 mL centrifuge tube is placed in a horizontal centrifuge and centrifuged at 300 g for 30 minutes. An acceleration time of the horizontal centrifuge is adjusted to reach a maximum speed within 2 minutes, and deceleration is set to be slow for optimal gradient centrifugation effect.

\textcircled{5}. After centrifugation, four layers in the 50 mL centrifuge tube are observed: a top layer is plasma and PBS, a middle layer is a white, cloudy mononuclear cell layer, followed by a Ficoll separation solution layer, and a bottom layer is neutrophils and red blood cells.

\textcircled{6}. The white, cloudy mononuclear cell layer is carefully aspirated using a Pasteur pipette and transferred to a 15 mL centrifuge tube.

\textcircled{7}. 1×PBS solution with a volume three times that of the mononuclear cells is added to the 15 mL centrifuge tube for washing, followed by centrifugation at 1000 rpm for 5 minutes, and supernatant is discarded.

\textcircled{8}. The step 7 is repeated once to obtain a pellet. The pellet is the PBMCs, which are ready for subsequent experiments.

In a population of patients with the coronary heart disease, an expression level of the BOB1-seRNA is studied in relation to Genisin score and SYNTAX score by the qPCR experiment. It is found to be significantly negatively correlated with coronary heart disease risk. That is, the higher the expression level of the BOB1-seRNA, the lower the risk of coronary artery lesions in patients (as shown in A and B in FIG. 4).

Apparently, the above embodiments of the disclosure are merely illustrative embodiments provided to clearly explain the disclosure and are not intended to limit the scope of the disclosure. Based on the above description, those skilled in the art can make various other forms of modifications, variations, or improvements. It is neither necessary nor possible to exhaustively list all possible embodiments here. Any modifications, equivalent substitutions, and improvements made within the spirit and principles of the disclosure should be included within the scope of protection of the appended claims.

What is claimed is:

1. A B-cell specific octamer-binding protein 1 super-enhancer ribonucleic acid (BOB1-seRNA), wherein the nucleotide sequence of the BOB1-seRNA is shown in SEQ ID NO: 1.

2. An application method of the BOB1-seRNA as claimed in claim 1, comprising:
preparing a drug for preventing, alleviating, or treating atherosclerosis by using the BOB1-seRNA.

3. A drug for preventing, alleviating or treating atherosclerosis, wherein an active ingredient of the drug comprises a substance that promotes an expression of a BOB1-seRNA.

4. The drug as claimed in claim 3, wherein the substance that promotes the expression of the BOB1-seRNA comprises a recombinant expression vector for overexpressing the BOB1-seRNA, a super-expression vector, an interference vector, a recombinant virus, or a recombinant gene expression kit.

5. The drug as claimed in claim 3, wherein the drug further comprises a pharmaceutically acceptable excipient.

6. The drug as claimed in claim 3, wherein a dosage form of the drug comprises an oral preparation, a topical preparation, an injectable preparation, or a molecular reagent kit.

7. An application method of the BOB1-seRNA as claimed in claim 1, comprising:
preparing a product for predicting a risk of coronary heart disease.


SEQUENCE LISTING (1 sequence)

SEQ ID NO: 1 - DNA, 469 bp, synthetic construct
  source 1..469: mol_type = other DNA
agctcagggccctccgaacaaagcagcgctcggtgccggacctgcagctctccccacccggcagcagcgccctcagcacgcccgggaacccccgacccctctgagcccggggtactgcgcccgggtctccacgcccagagatgctccccggtctccaccgtcgggcaagccccaagcgcagcagcgcagagtcctggggtcaccagagctcgtactaggacatcgtctccccatttaacaccgcctccggtcccatctgagttgcaagtggtggggatgtggggctccggatcaaagtccccgaaaccgagcacttcccgaagcctccttggcctcgaaacaaaacaataacgcccaagcgacctactgccttggagattcccagcctgagctccccaggtccctcagaggacccgagaggaacatcttactgcttctcttcataaattgagaggaaggaggccacaaa